SEQUENCE LISTING (26 sequences)

SEQ ID NO: 1 - protein, 4 aa, unidentified
  REGION 1..4: note = Description of Unknown: Bovine sequence
  MOD_RES 1..3: note = May or may not be phosphorylated
SSSE

SEQ ID NO: 2 - protein, 5 aa, unidentified
  REGION 1..5: note = Description of Unknown: Bovine sequence
  MOD_RES 1..3: note = May or may not be phosphorylated
SSSEE

SEQ ID NO: 3 - protein, 5 aa, unidentified
  REGION 1..5: note = Description of Unknown: Bovine sequence
  MOD_RES 1: note = phosphorylated serine
  MOD_RES 2: note = phosphorylated serine
  MOD_RES 3: note = phosphorylated serine
SSSEE

SEQ ID NO: 4 - protein, 25 aa, unidentified
  REGION 1..25: note = Description of Unknown: Bovine sequence
  MOD_RES 15: note = phosphorylated serine
  MOD_RES 17: note = phosphorylated serine
  MOD_RES 18: note = phosphorylated serine
  MOD_RES 19: note = phosphorylated serine
RELEELNVPGEIVESLSSSEESITR

SEQ ID NO: 5 - protein, 21 aa, unidentified
  REGION 1..21: note = Description of Unknown: Bovine sequence
  MOD_RES 8: note = phosphorylated serine
  MOD_RES 9: note = phosphorylated serine
  MOD_RES 10: note = phosphorylated serine
QMEAESISSSEEIVPDSVEQK

SEQ ID NO: 6 - protein, 21 aa, unidentified
  REGION 1..21: note = Description of Unknown: Bovine sequence
  MOD_RES 8: note = phosphorylated serine
  MOD_RES 9: note = phosphorylated serine
  MOD_RES 10: note = phosphorylated serine
KNTMEHVSSSEESIISNETYK

SEQ ID NO: 7 - protein, 17 aa, unidentified
  REGION 1..17: note = Description of Unknown: Bovine sequence
  MOD_RES 11: note = phosphorylated serine
  MOD_RES 13: note = phosphorylated serine
KVNELSKNIGSESTEDQ

SEQ ID NO: 8 - protein, 20 aa, unidentified
  REGION 1..20: note = Description of Unknown: Bovine sequence
  MOD_RES 9: note = phosphorylated serine
  MOD_RES 11: note = phosphorylated serine
PTLNREQLSTSEENSKKTVD

SEQ ID NO: 9 - protein, 24 aa, unidentified
  REGION 1..24: note = Description of Unknown: Bovine sequence
  MOD_RES 14: note = phosphorylated serine
  MOD_RES 16: note = phosphorylated serine
  MOD_RES 17: note = phosphorylated serine
  MOD_RES 18: note = phosphorylated serine
ELEELNVPGEIVESLSSSEESITR

SEQ ID NO: 10 - protein, 24 aa, unidentified
  REGION 1..24: note = Description of Unknown: Bovine sequence
  MOD_RES 14: note = phosphorylated serine
  MOD_RES 16: note = phosphorylated serine
  MOD_RES 17: note = phosphorylated serine
  MOD_RES 18: note = phosphorylated serine
RELEELNVPGEIESLSSSEESITR

SEQ ID NO: 11 - protein, 21 aa, unidentified
  REGION 1..21: note = Description of Unknown: Bovine sequence
  MOD_RES 6: note = phosphorylated serine
  MOD_RES 8: note = phosphorylated serine
  MOD_RES 9: note = phosphorylated serine
  MOD_RES 10: note = phosphorylated serine
  MOD_RES 17: note = phosphorylated serine
QMEAESISSSEEIVPNSVEQK

SEQ ID NO: 12 - protein, 21 aa, unidentified
  REGION 1..21: note = Description of Unknown: Bovine sequence
  MOD_RES 8: note = phosphorylated serine
  MOD_RES 9: note = phosphorylated serine
  MOD_RES 10: note = phosphorylated serine
  MOD_RES 16: note = phosphorylated serine
KNTMEHVSSSEESIISQETYK

SEQ ID NO: 13 - protein, 17 aa, unidentified
  REGION 1..17: note = Description of Unknown: Bovine sequence
  MOD_RES 11: note = phosphorylated serine
  MOD_RES 13: note = phosphorylated serine
KVNELSKDIGSESTEDQ

SEQ ID NO: 14 - protein, 129 aa, unidentified
  REGION 1..129: note = Description of Unknown: Bovine sequence
  MOD_RES 5: note = phosphorylated serine
  MOD_RES 45: note = phosphorylated serine
  MOD_RES 46: note = phosphorylated serine
  MOD_RES 47: note = phosphorylated serine
  MOD_RES 50: note = phosphorylated serine
  MOD_RES 118: note = phosphorylated serine
  MOD_RES 120: note = phosphorylated serine
ESIISQETYKQEKNMAINPSKENLCSTFCKEVVRNANEEETSIGSSSEESAEVATEEVKITVDDKHYQKALNEINQFYQKFPGYLQYLYQGPIVLNPWNQVLRNAVPITPTLNREQLSTSEENSKKTVN

SEQ ID NO: 15 - protein, 23 aa, unidentified
  REGION 1..23: note = Description of Unknown: Bovine sequence
  MOD_RES 13: note = phosphorylated serine
  MOD_RES 15: note = phosphorylated serine
  MOD_RES 16: note = phosphorylated serine
  MOD_RES 17: note = phosphorylated serine
ELEELNVPGEIESLSSSEESITR

SEQ ID NO: 16 - protein, 7 aa, unidentified
  REGION 1..7: note = Description of Unknown: Bovine sequence
  MOD_RES 1..2: note = A positively charged amino acid or absent
  SITE 1..2: note = This region may encompass 0-2 residues
  MOD_RES 3: note = phosphorylated serine
  MOD_RES 4: note = phosphorylated serine
  MOD_RES 5: note = phosphorylated serine
  MOD_RES 6..7: note = A positively charged amino acid or absent
  MOD_RES 6..7: note = This region may encompass 0-2 residues
XXSSSXK

SEQ ID NO: 17 - protein, 5 aa, unidentified
  REGION 1..5: note = Description of Unknown: Bovine sequence
  MOD_RES 3: note = phosphorylated serine
  MOD_RES 4: note = phosphorylated serine
  MOD_RES 5: note = phosphorylated serine
KKSSS

SEQ ID NO: 18 - protein, 6 aa, unidentified
  REGION 1..6: note = Description of Unknown: Bovine sequence
  MOD_RES 3: note = phosphorylated serine
  MOD_RES 4: note = phosphorylated serine
  MOD_RES 5: note = phosphorylated serine
KKSSSK

SEQ ID NO: 19 - protein, 7 aa, unidentified
  REGION 1..7: note = Description of Unknown: Bovine sequence
  MOD_RES 3: note = phosphorylated serine
  MOD_RES 4: note = phosphorylated serine
  MOD_RES 5: note = Phosphorylated serine
KKSSSKK

SEQ ID NO: 20 - protein, 6 aa, unidentified
  REGION 1..6: note = Description of Unknown: Bovine sequence
  MOD_RES 2: note = phosphorylated serine
  MOD_RES 3: note = phosphorylated serine
  MOD_RES 4: note = phosphorylated serine
KSSSKK

SEQ ID NO: 21 - protein, 5 aa, unidentified
  REGION 1..5: note = Description of Unknown: Bovine sequence
  MOD_RES 2: note = phosphorylated serine
  MOD_RES 3: note = phosphorylated serine
  MOD_RES 4: note = phosphorylated serine
KSSSK

SEQ ID NO: 22 - protein, 4 aa, unidentified
  REGION 1..4: note = Description of Unknown: Bovine sequence
  MOD_RES 2: note = phosphorylated serine
  MOD_RES 3: note = phosphorylated serine
  MOD_RES 4: note = phosphorylated serine
KSSS

SEQ ID NO: 23 - protein, 5 aa, unidentified
  REGION 1..5: note = Description of Unknown: Bovine sequence
  MOD_RES 1: note = phosphorylated serine
  MOD_RES 2: note = phosphorylated serine
  MOD_RES 3: note = phosphorylated serine
SSSKK

SEQ ID NO: 24 - protein, 4 aa, unidentified
  REGION 1..4: note = Description of Unknown: Bovine sequence
  MOD_RES 1: note = phosphorylated serine
  MOD_RES 2: note = phosphorylated serine
  MOD_RES 3: note = phosphorylated serine
SSSK

SEQ ID NO: 25 - protein, 5 aa, unidentified
  REGION 1..5: note = Description of Unknown: Bovine sequence
  MOD_RES 3: note = phosphorylated serine
  MOD_RES 4: note = phosphorylated serine
  MOD_RES 5: note = phosphorylated serine
KKSSS

SEQ ID NO: 26 - protein, 28 aa, unidentified
  REGION 1..28: note = Description of Unknown: Bovine sequence
  MOD_RES 15: note = phosphorylated serine
  MOD_RES 17: note = phosphorylated serine
  MOD_RES 18: note = phosphorylated serine
  MOD_RES 19: note = phosphorylated serine
RELEELNVPGEIVESLSSSEESITRINK

(19) United States
(12) Patent Application Publication
(10) Pub. No.: US 2026/0314637 A1
(43) Pub. Date: Oct. 15, 2026

(54) ENHANCED MILK QUALITY

(71) Applicant: MILEUTIS LTD., Ness Ziona (IL)

(72) Inventors: Jose Mario ISCOVICH, Gan Yavne (IL); Javier ISCOVICH, Tel Aviv (IL)

(73) Assignee: MILEUTIS LTD., Ness Ziona (IL)

(21) Appl. No.: 19/483,983

(22) PCT Filed: May 16, 2024
(86) PCT No.: PCT/IL2024/050486

§ 371 Date: Nov. 13, 2025

Related U.S. Application Data
(60) Provisional application No. 63/502,462, filed on May 16, 2023.

(51) Int. Cl. A23K 20/147; A23J 3/10; A23K 50/10; A61K 38/17; A61P 3/02

(52) U.S. Cl. CPC A23K 20/147; A23J 3/10; A23K 50/10; A61K 38/1709; A61P 3/02

(57) ABSTRACT

The current disclosure relates to a method of increasing milk quality in lactating mammals comprising administering a composition comprising at least one casein derived peptide.

### SEQUENCE LISTING STATEMENT

The instant application contains a Sequence Listing which has been submitted electronically in ASCII format and is hereby incorporated by reference in its entirety. Said ASCII copy, created on May 16, 2024, is named P-625310-PC-ST26 and is 69,632 bytes in size.

### BACKGROUND OF THE INVENTION

The casein protein comprises three fractions, α, β and κ, according to their electrophoretic mobility. Casein hydrolysate is the hydrolyzed form of casein which includes, among others, the active beta-casein-derived peptide. It has been established that casein hydrolysate plays a role in immune responses against microbial and viral infections.

The global dairy market is estimated at more than $500 billion with an average annual growth rate of 4%. Bovine milk attributes a significant portion of the market whereas plant-based alternatives account for $1 billion in the US and an estimated $700 million is estimated for lactose-intolerant milk. Mammal- or mammalian-produced milk is a very complex fluid that includes several thousand components. Mammal- or mammalian-produced milk includes water, a variety of different lipids, sugar, a variety of different proteins, and a variety of different inorganic salts and compounds.

Dietary protein is an essential nutrient for human health and growth. The World Health Organization recommends that dietary protein should contribute approximately 10 to 15% of energy intake when in energy balance and weight stable. Average daily protein intakes in various countries indicate that these recommendations are consistent with the amount of protein being consumed worldwide. Meals with an average of 20 to 30% of energy from protein are representative of high-protein diets when consumed in energy balance.

The body cannot synthesize certain amino acids that are necessary for health and growth, and instead must obtain them from food. These amino acids, called “essential amino acids”, are Histidine (H), Isoleucine (I), Leucine (L), Lysine (K), Methionine (M), Phenylalanine (F), Threonine (T), Tryptophan (W), and Valine (V). Dietary proteins that provide all the essential amino acids are referred to as “high quality” proteins. Animal foods such as meat, fish, poultry, eggs, and dairy products are generally regarded as high-quality protein sources that provide a good balance of essential amino acids. Casein proteins and whey (the protein in the liquid that remains after milk has been curdled and strained) are major sources of high-quality dietary protein. Foods that do not provide a good balance of essential amino acids are referred to as “low quality” proteins. Most fruits and vegetables are poor sources of protein. Some plants foods including beans, peas, lentils, nuts and grains (such as wheat) are better sources of protein. Soy, a vegetable protein manufactured from soybeans, is considered by some to be a high-quality protein.

Raw milk composition and properties are crucial for the control of the dairy product quality. There is still a great need for sources of high-quality dietary products.

### SUMMARY OF THE INVENTION

In some aspects, disclosed herein is a method for increasing milk quality in lactating mammals comprising administering a composition comprising at least one casein derived peptide.

In some related aspects, the increased milk quality comprises increased milk protein, increased milk butterfat, increased milk lactose, increased energy corrected milk (ECM) or any combination thereof.

In some related aspects, the milk protein percentage in the milk is in the range of 2.5%-6.5%. In some further related aspects, the milk protein percentage in the milk is increased by 0.5%-30% in comparison to milk protein percentage in control lactating mammals. A person of ordinary skilled in the art will understand the phrase “control lactating mammals” as mammals that were not administered any treatment, or which were administered treatment different from a composition comprising at least one casein derived peptide.

In some related aspects, the milk butterfat percentage in the milk is in the range of 2.5%-6.5%. In some further related aspects, the milk butterfat percentage in the milk is increased by 0.5-%-30% in comparison to milk butterfat percentage in control lactating mammals.

In some related aspects, the milk lactose percentage in the milk is in the range of 3.5%-7.8%. In some further related aspects, the milk lactose percentage in the milk is increased by 0.5%-30% in comparison to milk lactose percentage in control lactating mammals.

In some related aspects, the ECM is increased by 0.5%-20% in comparison to ECM in control lactating mammals.

In some embodiments, the milk quality is increased in any stage of the lactating cycle.

In some embodiments, the administration is at the beginning of the dry period. In some embodiments, the milk quality is increased, independent of the dry period length.

In some related aspects, the casein derived peptide comprises natural peptide, synthetic peptide, semi-synthetic peptide, or any combination thereof.

In some further related aspects, the casein derived peptide comprises one or more fragments of β-casein, αS1-casein, αS2-casein and κ-casein. In some further related aspects, the casein derived peptide further comprises amino acids with different lengths.

In some further related aspects, the casein derived peptide comprises a casein hydrolysate. In some further related aspects, the casein derived peptide comprises a phosphopeptide. In some further related aspects, the phosphopeptide comprises an amino acid sequence selected form the group consisting of SEQ ID NO. 1-SEQ ID NO. 26.

In some aspects, the composition is free of antimicrobials and comprises an acceptable carrier.

In some aspects, the method comprises administrating between 10 ng/ml to 500 mg/ml of the casein derived peptide.

In some aspects, the administration comprises intramammary infusion to a single teat or a plurality of teats.

In some aspects, the milk is essentially free of residues and can be used as raw milk, for dairy production, for breastfeeding, bakery, confectionary, feeding or any combination thereof. In some further aspects, the dairy product comprises milk, whey, yogurt, cheese, cream, butter, milk drinks with high protein or combination thereof.

In some aspects, disclosed herein is a composition comprising at least one casein derived peptide for use in increasing milk quality in lactating mammals.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a graphical illustration of day/average energy corrected milk (ECM) production (Kg) in dairy cows at the dry-off stage, treated with bovine casein hydrolysate (bCNH) along 305 days of subsequent lactation. Control-cows were treated with antibiotics. bCNH11—cows were treated with single dose of bovine casein hydrolysate (bCNH). bCNH22—cows were treated with two doses of bovine casein hydrolysate (bCNH). *p<0.05. control: 36.67, bCNH11: 39.83, bCNH 22:39.62;

FIG. 2 is a graphical illustration, by month, of energy corrected milk (ECM) production (Kg) in dairy cows at the dry-off stage, treated with bovine casein hydrolysate (bCNH) along 305 days of subsequent lactation. Control—cows were treated with antibiotics. bCNH11—cows were treated with single dose of bovine casein hydrolysate (bCNH). bCNH22—cows were treated with two doses of bovine casein hydrolysate (bCNH). *p<0.05;

FIG. 3 is a graphical illustration of day/average butterfat yield (kg) in dairy cows at the dry-off stage, treated with bovine casein hydrolysate (bCNH) along 305 days of subsequent lactation. Control-cows were treated with antibiotics. bCNH11—cows were treated with single dose of bovine casein hydrolysate (bCNH). bCNH22—cows were treated with two doses of bovine casein hydrolysate (bCNH). *p<0.05 bCNH11: 1.45, bCNH22: 1.49;

FIG. 4 is a graphical illustration, by month, of butterfat yield (kg) in dairy cows at the dry-off stage, treated with bovine casein hydrolysate (bCNH) along 305 days of subsequent lactation. Control—cows were treated with antibiotics. bCNH11—cows were treated with single dose of bovine casein hydrolysate (bCNH). bCNH22—cows were treated with two doses of bovine casein hydrolysate (bCNH). *p<0.05;

FIG. 5 is a graphical illustration of day/average milk protein yield (kg) in dairy cows at the dry-off stage, treated with bovine casein hydrolysate (bCNH) along 305 days of subsequent lactation. Control—cows were treated with antibiotics. bCNH11—cows were treated with single dose of bovine casein hydrolysate (bCNH). bCNH22—cows were treated with two doses of bovine casein hydrolysate (bCNH). *p<0.05. Control 1.23, bCNH11: 1.31, bCNH22: 1.36;

FIG. 6 is a graphical illustration, by month, of milk protein yield (kg) in dairy cows at the dry-off stage, treated with bovine casein hydrolysate (bCNH) along 305 days of subsequent lactation. Control—cows were treated with antibiotics. bCNH11—cows were treated with single dose of bovine casein hydrolysate (bCNH). bCNH22—cows were treated with two doses of bovine casein hydrolysate (bCNH). *p<0.05;

FIG. 7 is a graphical illustration of day/average milk lactose yield (kg) in dairy cows at the dry-off stage, treated with bovine casein hydrolysate (bCNH) along 305 days of subsequent lactation. Control—cows were treated with antibiotics. bCNH11—cows were treated with single dose of bovine casein hydrolysate (bCNH). bCNH22—cows were treated with two doses of bovine casein hydrolysate (bCNH). **p<0.01. Control 1.91 (SE) bCNH11: 1.94, bCNH22: 1.97;

FIG. 8 is a graphical illustration, by month, of milk lactose yield (kg) in dairy cows at the dry-off stage, treated with bovine casein hydrolysate (bCNH) along 305 days of subsequent lactation. Control—cows were treated with antibiotics. bCNH11—cows were treated with single dose of bovine casein hydrolysate (bCNH). bCNH22—cows were treated with two doses of bovine casein hydrolysate (bCNH). **p<0.01;

FIG. 9 is a graphical illustration, by month, of ECM. Average of monthly ECM before entry to the trial was used as a covariate. Energy corrected milk yield of control group for the whole period of post-milking reinitiation was 35.88 (SE-4.06) and of bCNH group was 39.86 (SE=2.76), *P=0.43;

FIG. 10 is a graphical illustration, by month, of Protein Values (kg). Average of monthly protein yield before entry to the trial was used as a covariate. The protein yield of control group for the whole 3 months period after initiation of milking was 1.32 kg (SE=0.098) and of bCNH group—1.38 kg (SE=0.065), P=0.08;

FIG. 11 is a graphical illustration, by month, of Butterfat Values (kg). Average of monthly butterfat yield before entry to the trial was used as a covariate. Butterfat yield of control group was 1.36 kg (SE=0.14) and of bCNH group—1.49 (SE=0.09), (*P=0.46);

FIG. 12 is a graphical illustration, by month, of Lactose Values (kg). Average of monthly lactose yield before entry to the trial was used as a covariate. Least Square mean of lactose yield of control group was 1.82 (SE=0.18) and of bCNH group—1.90 (SE=0.12);

FIG. 13 is a graphical illustration, by month, of Composite-Bulk Milk Somatic Cell Count (BMSCC) During the Pre- and Post-Re-Milking Periods. Bulk milk somatic cell count values are not normally distributed (Prob>chiSq=0.026). Therefore, BMSCC values were transformed to log 10. The differences in each Month-In-Milk (MIM) revealed that log 10BMSCC of control cows was significantly higher than that of experimental group (*P=0.005) in the first month post milking initiation;

FIG. 14 is a graphical illustration, by month, of Monthly Milk Yield Values (kg). Average of monthly milk yield before entry to the trial was used as a covariate. Monthly milk yield of control group for the whole period of post-milking reinitiation was 39.27 kg (SE=3.56) per day on average, and of bCNH group—39.92 kg (SE=2.37, P=0.88) per day on average.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

### Method of Increasing Milk Quality

In some embodiments, disclosed herein is a method for increasing milk quality in lactating mammals comprising administering a composition comprising at least one casein derived peptide.

In some embodiments, the increased milk quality comprises increased milk protein, increased milk butterfat, increased milk lactose, increased energy corrected milk (ECM) or any combination thereof. In one embodiment, the increased milk quality comprises increased milk protein. In another embodiment, the increased milk quality comprises increased milk butterfat. In another embodiment, the increased milk quality comprises increased milk lactose. In another embodiment, the increased milk quality comprises increased energy corrected milk (ECM). In another embodiment, the increased milk quality comprises increased milk protein and increased milk butterfat. In another embodiment, the increased milk quality comprises increased milk protein and increased milk lactose. In another embodiment, the increased milk quality comprises increased milk protein and increased ECM. In another embodiment, the increased milk quality comprises increased milk butterfat and increased milk lactose. In another embodiment, the increased milk quality comprises increased milk butterfat and increased ECM. In another embodiment, the increased milk quality comprises increased milk lactose and increased ECM. In another embodiment, the increased milk quality comprises increased milk protein, increased milk butterfat, increased milk lactose and increased energy corrected milk (ECM).

A skilled artisan would understand that energy corrected milk (ECM) determines the amount of energy in the milk based upon the milk, fat and protein included. The determination of the ECM can be performed, for example, by following the equation: ECM (kg)=Milk (kg)*0.1+Fat (%)*Milk (kg)*10+Protein (%)*Milk (kg)*15.8.

In one embodiment, disclosed herein, is a method for increasing milk protein in lactating mammals comprising administering a composition comprising at least one casein derived peptide. In one embodiment, the milk protein is extracted from the milk.

In one embodiment, disclosed herein is a method for increasing milk butterfat in lactating mammals comprising administering a composition comprising at least one casein derived peptide. In one embodiment, the milk butterfat is extracted from the milk.

In one embodiment, disclosed herein is a method for increasing milk lactose in lactating mammals comprising administering a composition comprising at least one casein derived peptide. In one embodiment, the milk lactose is extracted from the milk.

In one embodiment, disclosed herein is a method for increasing ECM in lactating mammals comprising administering a composition comprising at least one casein derived peptide.

In some embodiments the milk protein percentage in the milk is in the range of 2.5%-6.5%. In one embodiment, the milk protein percentage in the milk is 2.5%. In another embodiment, the milk protein percentage in the milk is 3.0%. In another embodiment, the milk protein percentage in the milk is 3.5%. In another embodiment, the milk protein percentage in the milk is 4.0%. In another embodiment, the milk protein percentage in the milk is 4.5%. In another embodiment, the milk protein percentage in the milk is 5.0%. In another embodiment, the milk protein percentage in the milk is 5.5%. In another embodiment, the milk protein percentage in the milk is 6.0%. In another embodiment, the milk protein percentage in the milk is 6.5%.

In some embodiments the milk protein percentage in the milk is increased by 0.5%-30.0% in comparison to milk protein in control lactating mammals. In some embodiments, the milk is increased by 1.0%-20.0% in comparison to milk protein in control lactating mammals. In some embodiments, the milk is increased by 3.0%-10.0% in comparison to milk protein in control lactating mammals. In some embodiments, the milk is increased by 5.0%-8.0% in comparison to milk protein in control lactating mammals.

In one embodiment, the milk protein percentage in the milk is increased by 0.5% in comparison to milk protein in control lactating mammals. In another embodiment, the milk protein percentage in the milk is increased by 1.0% in comparison to milk protein in control lactating mammals. In another embodiment, the milk protein percentage in the milk is increased by 5.0% in comparison to milk protein in control lactating mammals. In another embodiment, the milk protein percentage in the milk is increased by 10.0% in comparison to milk protein in control lactating mammals. In another embodiment, the milk protein percentage in the milk is increased by 15.0% in comparison to milk protein in control lactating mammals. In another embodiment, the milk protein percentage in the milk is increased by 20.0% in comparison to milk protein in control lactating mammals. In another embodiment, the milk protein percentage in the milk is increased by 25.0% in comparison to milk protein in control lactating mammals. In another embodiment, the milk protein percentage in the milk is increased by 30.0% in comparison to milk protein in control lactating mammals.

In some embodiments, the milk butterfat percentage in the milk is in the range of 2.5%-6.5%. In one embodiment, the milk butterfat percentage in the milk is 2.5%. In another embodiment, the milk butterfat percentage in the milk is 3.0%. In another embodiment, the milk butterfat percentage in the milk is 3.3%. In another embodiment, the milk butterfat percentage in the milk is 3.4%. In another embodiment, the milk butterfat percentage in the milk is 3.5%. In another embodiment, the milk butterfat percentage in the milk is 3.6%. In another embodiment, the milk butterfat percentage in the milk is 3.7%. In another embodiment, the milk butterfat percentage in the milk is 3.8%. In another embodiment, the milk butterfat percentage in the milk is 3.9%. In another embodiment, the milk butterfat percentage in the milk is 4.0%. In another embodiment, the milk butterfat percentage in the milk is 4.5%. In another embodiment, the milk butterfat percentage in the milk is 5.0%. In another embodiment, the milk butterfat percentage in the milk is 5.5%. In another embodiment, the milk butterfat percentage in the milk is 6.0%. In another embodiment, the milk butterfat percentage in the milk is 6.5%.

In some embodiments, the milk butterfat percentage in the milk is increased by 0.5%-30% in comparison to milk butterfat in control lactating mammals. In some embodiments, the milk butterfat percentage in the milk is increased by 1%-15% in comparison to milk butterfat in control lactating mammals. In some embodiments, the milk butterfat percentage in the milk is increased by 2%-6% in comparison to milk butterfat in control lactating mammals. In some embodiments, the milk butterfat percentage in the milk is increased by 3%-5% in comparison to milk butterfat in control lactating mammals.

In one embodiment, the milk butterfat percentage in the milk is increased by 0.5% in comparison to milk butterfat in control lactating mammals. In another embodiment, the milk butterfat percentage in the milk is increased by 1.0% in comparison to milk butterfat in control lactating mammals. In another embodiment, the milk butterfat percentage in the milk is increased by 5.0% in comparison to milk butterfat in control lactating mammals. In another embodiment, the milk butterfat percentage in the milk is increased by 10.0% in comparison to milk butterfat in control lactating mammals. In another embodiment, the milk butterfat percentage in the milk is increased by 15.0% in comparison to milk butterfat in control lactating mammals. In another embodiment, the milk butterfat percentage in the milk is increased by 20.0% in comparison to milk butterfat in control lactating mammals. In another embodiment, the milk butterfat percentage in the milk is increased by 25.0% in comparison to milk butterfat in control lactating mammals. In another embodiment, the milk butterfat percentage in the milk is increased by 30.0% in comparison to milk butterfat in control lactating mammals.

In some embodiments, the milk lactose percentage in the milk is in the range of 3.5%-7.8%. In one embodiment, the milk lactose percentage in the milk is 3.5%. In another embodiment, the milk lactose percentage in the milk is 4.0%. In another embodiment, the milk lactose percentage in the milk is 4.5%. In another embodiment, the milk lactose percentage in the milk is 5.0%. In another embodiment, the milk lactose percentage in the milk is 5.5%. In another embodiment, the milk lactose percentage in the milk is 6.0%. In another embodiment, the milk lactose percentage in the milk is 6.5%. In another embodiment, the milk lactose percentage in the milk is 7.0%. In another embodiment, the milk lactose percentage in the milk is 7.5%. In another embodiment, the milk lactose percentage in the milk is 7.8%.

In some embodiments, the milk lactose percentage in the milk is increased by 0.5%-30% in comparison to milk lactose in control lactating mammals. In some embodiments, the milk lactose percentage in the milk is increased by 1%-15% in comparison to milk lactose in control lactating mammals. In some embodiments, the milk lactose percentage in the milk is increased by 2%-6% in comparison to milk lactose in control lactating mammals. In some embodiments, the milk lactose percentage in the milk is increased by 3%-5% in comparison to milk lactose in control lactating mammals.

In one embodiment, the milk lactose percentage in the milk is increased by 0.5% in comparison to milk lactose in control lactating mammals. In another embodiment, the milk lactose percentage in the milk is increased by 1% in comparison to milk lactose in control lactating mammals. In another embodiment, the milk lactose percentage in the milk is increased by 1.5% in comparison to milk lactose in control lactating mammals. In another embodiment, the milk lactose percentage in the milk is increased by 2.0% in comparison to milk lactose in control lactating mammals. In another embodiment, the milk lactose percentage in the milk is increased by 2.5% in comparison to milk lactose in control lactating mammals. In another embodiment, the milk lactose percentage in the milk is increased by 3.0% in comparison to milk lactose in control lactating mammals. In another embodiment, the milk lactose percentage in the milk is increased by 3.5% in comparison to milk lactose in control lactating mammals. In another embodiment, the milk lactose percentage in the milk is increased by 4.0% in comparison to milk lactose in control lactating mammals. In another embodiment, the milk lactose percentage in the milk is increased by 4.5% in comparison to milk lactose in control lactating mammals. In another embodiment, the milk lactose percentage in the milk is increased by 5.0% in comparison to milk lactose in control lactating mammals. In another embodiment, the milk lactose percentage in the milk is increased by 5.5% in comparison to milk lactose in control lactating mammals. In another embodiment, the milk lactose percentage in the milk is increased by 6.0% in comparison to milk lactose in control lactating mammals.

In another embodiment, the milk lactose percentage in the milk is increased by 10% in comparison to milk lactose in control lactating mammals. In another embodiment, the milk lactose percentage in the milk is increased by 15% in comparison to milk lactose in control lactating mammals. In another embodiment, the milk lactose percentage in the milk is increased by 20% in comparison to milk lactose in control lactating mammals. In another embodiment, the milk lactose percentage in the milk is increased by 25% in comparison to milk lactose in control lactating mammals. In another embodiment, the milk lactose percentage in the milk is increased by 30% in comparison to milk lactose in control lactating mammals.

In some embodiments, the ECM is increased by 0.5%-20% in comparison to ECM in control lactating mammals. In some embodiments, the ECM is increased by 2.0%-15% in comparison to ECM in control lactating mammals. In some embodiments, the ECM is increased by 5.0%-10% in comparison to ECM in control lactating mammals. In some embodiments, the ECM is increased by 7.5%-9% in comparison to ECM in control lactating mammals.

In some embodiments, the milk quality further comprises decrease in somatic cell count (SCC). In one embodiment, the decrease in SCC is after milking reinitiation. In one embodiment, the decrease in SCC is in treated subjects, in comparison to untreated subjects.

In some embodiments, the milk quality is increased at any stage of the lactating cycle. A person of ordinary skilled in the art would understand that “lactating cycles” means the period between one calving and the next. The cycle is split into phases, the early lactation, mid lactation, late lactation, and the dry period.

In one embodiment, the milk quality is increased at early lactation, mid lactation, late lactation, or any combination thereof. In another embodiment, the milk quality is increased at early lactation. In another embodiment, the milk quality is increased at mid lactation. In another embodiment, the milk quality is increased at late lactation. In another embodiment, the milk quality is increased at early lactation and mid lactation. In another embodiment, the milk quality is increased at early lactation and late lactation. In another embodiment, the milk quality is increased at mid lactation and late lactation. In another embodiment, the milk quality is increased at early lactation, mid lactation, and late lactation.

In some embodiments, the milk quality is increased during the same lactation of administration of said composition comprising at least one casein derived peptide. In some embodiments, the milk quality is increased during the following lactation of administration of said composition comprising at least one casein derived peptide.

In some embodiments, the administration is at the beginning of the dry period. In some embodiments, the administration is during the lactation period.

A skilled artisan would understand that “dry period” means the period before calving that cows are not milked, which is currently about 6 to 9 weeks. The dry period has multiple functions. Main functions are to allow the cow a rest period before birth of the next calf and to maximize milk yield in the next lactation. During the dry period, mammary cells renew at a faster rate than when cows would be milked up to calving. At the beginning of the dry period many dairy farmers administer antibiotics in order to, among other purposes, treat the cow in case of persistent subclinical mastitis.

In some embodiments, the milk quality is increased, independent of the dry period length. In one embodiment, the dry off period comprises 28 to 120 days. In another embodiment, the dry off period comprises 28 to 100 days. In another embodiment, the dry off period comprises 28 to 80 days. In another embodiment, the dry off period comprises 28 to 60 days. In another embodiment, the dry off period comprises 28 to 40 days. In another embodiment, the dry off period comprises 40 to 49 days. In another embodiment, the dry off period comprises 50 to 59 days.

In some embodiments, the milk is essentially free of residues.

In some embodiments, the milk can be used as raw milk, for dairy production, for breastfeeding, bakery, confectionary, feeding or any combination thereof. In one embodiment, the milk can be used as raw milk. In another embodiment, the milk can be used for dairy production. In another embodiment, the milk can be used for breastfeeding. In another embodiment, the milk can be used for bakery. In another embodiment, the milk can be used for confectionary. In another embodiment, the milk can be used for feeding.

In another embodiment, the milk can be used for extraction of protein. In another embodiment, the milk can be used for extraction of butterfat. In another embodiment, the milk can be used for extraction of lactose.

In one embodiment, the dairy product comprises milk, whey, yogurt, cheese, cream, butter, milk drinks with high protein or combination thereof. In another embodiment, the dairy product comprises milk. In another embodiment, the dairy product comprises whey. In another embodiment, the dairy product comprises yogurt. In another embodiment, the dairy product comprises cheese. In another embodiment, the dairy product comprises cream. In another embodiment, the dairy product comprises butter. In another embodiment, the dairy product comprises milk drinks with high protein.

In some embodiments, the dairy product comprises a high percentage of protein.

In one embodiment, the product resulting from the high-quality milk, is for use in the treatment of disease. In one embodiment, the product resulting from the high-quality milk, is for use in building and repairing muscle, skin, building and repairing other body tissues, treating infection, balancing body fluids, and carrying oxygen through the body, supporting cancer treatment, or any combination thereof.

In some embodiments, the product resulting from the high-quality milk, can be used for applications in the non-food area. In one embodiment, the non-food area comprises manufacture of plastic and other solid materials, textile fibers, glues, in the production of ethanol or methane, in the research area as barriers to non-polar substances such as oxygen, carbon dioxide and aromas, use in several technical applications such as protective coating and foams, paper coating, adhesives or injection molding disposables, as emulsifiers, as detergents, drug delivery, or any combination thereof.

In some embodiments, disclosed herein is a composition comprising at least one casein derived peptide for use in increasing milk quality in lactating mammals.

### Casein Peptides

In some embodiments, the method as described comprises administering a composition comprises at least one casein derived peptide.

Casein is a protein in non-human mammal's milk, also found in human mammal's milk known to include the subgroups αS1, αS2, β and κ. Casein is defined according to the amino acid sequences of each of the subgroups αS1, αS2, β and κ. In the context of the present disclosure, when referring to casein, it is to be understood as also including acid casein, salts of casein, phosphorous containing casein and rennet casein.

The term “protein” as used herein refers to amino acid residues, connected by peptide bonds. A protein sequence is generally reported from the N-terminal end containing free amino group to the C-terminal end containing free carboxyl group. Amino acids, as used herein, refer to naturally occurring and synthetic amino acids, as well as amino acid analogs, and amino acid mimetics, that function in a manner similar to the naturally occurring amino acids. Amino acids may be referred to herein by either their commonly known three letter symbols or by the one-letter symbols recommended by the IUPAC-IUB Biochemical Nomenclature Commission.

The casein-derived peptide may be a single peptide or a mixture of different peptides which may be independently selected from a naturally occurring peptide, a semi-synthetic peptide, a synthetic peptide or a recombinant peptide. It should be further noted that the peptides according to the present disclosure may be produced synthetically, or by recombinant DNA technology, or by any other technology. Methods for producing peptides are well known in the art.

In some embodiments, the casein-derived peptide may comprise a casein protein breakdown product which occurs when casein protein is cleaved by enzymes or acids to peptide fragments (also known in the art by the term “casein hydrolysate”). A casein hydrolysate is to be understood as the hydrolyzed form of casein (protein). Casein hydrolysate includes, for example, the active beta-, alpha S1—, alpha S2—, kappa-casein-derived peptide known to those versed in the art. In some embodiments, the casein-derived peptide is or comprises a casein hydrolysate.

In some embodiments, the casein derived peptide comprises natural peptide, synthetic peptide, semi-synthetic peptide, or any combination thereof. In another embodiment, the casein derived peptide comprises a natural peptide. In another embodiment, the casein derived peptide comprises a synthetic peptide. In another embodiment, the casein derived peptide comprises a semi-synthetic peptide. In another embodiment, the casein derived peptide comprises a combination of natural peptide and synthetic peptide. In another embodiment, the casein derived peptide comprises a combination of natural peptide and semi-synthetic peptide. In another embodiment, the casein derived peptide comprises a combination of synthetic peptide and semi-synthetic peptide. In another embodiment, the casein derived peptide comprises a combination of natural peptide, synthetic peptide and semi-synthetic peptide.

Natural casein-derived peptides are typically obtained following enzymatic hydrolysis, the enzyme may be any mammal peptidase, such as, without being limited thereto, plasmin, pancreatin, trypsin, chymotrypsin, neutrase, alcalase, pepsin, carboxypeptidase, cathepsin, as well as plant peptidase such as, without being limited thereto, papain, bromelain, as well as enzymes from microorganism source. For example, a naturally occurring casein-derived peptide may be the result of an enzyme activity such as plasmin on casein subunits β-casein, αs1- and αs2-casein or κ-casein. In some embodiments, a casein hydrolysate is obtained by cleavage of the casein protein with trypsin.

A synthetic peptide may be obtained by any methods known in the art of peptide synthesis including chemical synthesis and recombinant DNA technology. For example, the peptides may be synthesized by using standard solid phase techniques.

In one embodiment, the synthetic peptide is a recombinant peptide.

A semi-synthetic casein-derived peptide may be obtained by chemical hydrolysis of casein, e.g. by prolonged boiling in a strong acid (acid-HVP) or strong base or using a chemical agent such as Cyanogen bromide (CNBr). The casein-derived peptide may also be obtained by molecular engineering, e.g. using recombinant DNA, in molecular techniques known in the art. In such an embodiment, the casein-derived peptide is a recombinant peptide.

In one embodiment, the recombinant peptide is produced by fermentation, tissue culture or combination thereof. In another embodiment, the recombinant peptide is produced by fermentation. In another embodiment, the recombinant peptide is produced by tissue culture. In another embodiment, the recombinant peptide is produced by a combination of fermentation and tissue culture.

In one embodiment, the tissue culture comprises mammary gland bovine tissue.

In some embodiments, the casein derived peptide comprises one or more fragments of β-casein, αS1-casein, αS2-casein, κ-casein or any combination thereof. In one embodiment, the casein derived peptide comprises one or more fragments of β-casein. In another embodiment, the casein derived peptide comprises one or more fragments of αS1-casein. In another embodiment, the casein derived peptide comprises one or more fragments of αS2-casein. In another embodiment, the casein derived peptide comprises one or more fragments of κ-casein. In another embodiment, the casein derived peptide comprises one or more fragments of combination of β-casein, αS1-casein, αS2-casein and κ-casein.

In one embodiment, the casein derived peptide further comprises amino acids with different lengths.

In one embodiment, the casein derived peptide comprises a casein hydrolysate.

In one embodiment, the casein derived peptide comprises a phosphopeptide.

As used herein, the term “phosphopeptide” designates a phosphorylated peptide in form of a conjugated peptide in which the non-peptide portion is a residue of phosphoric acid. The expression “phosphopeptide” or “phosphoserine” designates conjugated serine in which the non-peptide portion is a residue of phosphoric acid.

In some embodiments, the casein-derived peptide is a single peptide or mixture of a phosphopeptide, namely, which contains a single phosphorous group or is a phosphorus-enriched peptide. In some embodiments, the casein-derived peptide is any phosphoserine, phosphotyrosine, phosphothreonine, and/or phosphohystidine-enriched casein-derived peptides (casein phosphopeptide, CPP) and monovalent cation phosphocaseinates, such as sodium, potassium, calcium or ammonium phosphocaseinates.

In some embodiments, the casein-derived peptide is a phosphor-peptide.

The phosphor-peptide may be genetically engineered casein-derived peptides as well as peptidomimetics of casein-derived peptides. For example, phosphorylation of amino acids such as at least one serine residue may be performed by any method as is known in the art. The term “casein-derived peptide” also encompasses peptide fragments or peptidomimetic products obtained from or corresponding to one or more sections of casein protein. The peptidomimetic peptide may be for example a peptoid or a semipeptoid, which are peptide analogs, having, for example, modifications such as, but are not limited to, cyclization, N-terminus modification, C-terminus modification, peptide bond modification, including, but not limited to, CH₂—NH, CH₂—S, CH₂—S—O, O—C—NH, CH₂—O, CH₂—CH₂, S—C—NH, CH—CH or CF—CH, backbone modification and residue modification.

As used herein, the term “casein-derived peptide” further encompasses any derivatives, analogues, variants or homologues of any of the peptides. The term “derivative” is used to define amino acid sequences (peptide), with any insertions, deletions, substitutions, and modifications to the amino acid sequences (peptide) that do not alter the activity of the original peptides. By the term “derivative” it is also referred to homologues, variants and analogues thereof, as well as covalent modifications of polypeptides made according to the present invention.

In some embodiments, the modified, synthetic, semi-synthetic or other types of analogs of the naturally occurring casein-derived peptides are in some embodiments at least 75%, at times 85%, 90%, 95% and even 99% identical (in sequence) to a naturally occurring casein-derived peptide when the two sequences are optimally aligned. Further, any non-naturally occurring casein-derived peptide to be used in accordance with the present disclosure may retain at least part of the biological activity of the naturally occurring casein protein.

The present disclosure also encompasses homologues of the casein-derived peptide. The term “homologues” is used to define amino acid sequences (peptide) which maintain a minimal homology to the amino acid sequences defined by the invention, e.g. have at least about 65%, at least about 75%, at least about 85%, or at least about 95% overall sequence homology with the amino acid sequence of any of the peptide as structurally defined above, e.g. of a specified sequence.

In some embodiments, the casein-derived peptide may also include a chemical modification of a naturally occurring peptide, e.g. where one or more amino acids are deleted, substituted or modified, e.g. by removal of a side group, substitution of a side group or the introduction of a chemical group. Without being limited thereto, the chemical modification may include acetylation, acylation, amidation, ADP-ribosylation, glycosylation, GPI anchor formation, covalent attachment of a lipid or lipid derivative, methylation, myristoylation, pegylation, prenylation, phosphorylation, ubiquitination, or any similar process. When referring to replacement of an amino acid sequence by another, it is likely that the replacement is a conservative substitution. For example, one or more amino acid residues within a casein sequence is substituted by another amino acid of a similar polarity or charge. For example, the non-polar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Nonetheless, non-conservative substitutions may also take place as long as it does not significantly change the desired (casein like) biological activity of the resulting casein-derived peptide analog.

A casein-derived peptide in accordance with the present disclosure is characterized by a molecular weight of between about an average 100 to an average 10,000 Dalton (e.g. between 2 to 100 amino acids) at times between about an average 100 to an average 7,000 Dalton and at times between an average 1,000 to an average 5,000 Daltons.

A casein-derived peptide in accordance with the disclosure is characterized by a length of from 2 to 200, from 2 to 100 amino acids, at times between 4 amino acids to 40 amino acids, at times from 4 amino acids to 30 amino acids, at times 4 amino acids to 10 amino acids, at times between 10 amino acids to 50 amino acids.

In some embodiments, the casein-derived peptide comprises an amino acid sequence selected form the group consisting of SEQ ID NO. 1-SEQ ID NO. 26.

In one embodiment, the phosphopeptide comprises an amino acid sequence denoted as Ser-Ser-Ser-Glu (SEQ ID NO:1), wherein at least one Ser residue, at least two Ser residues or three Ser residues are phosphorylated (phosphorylated serine is denoted herein as Ser(p) or S(p)).

In another embodiment, the phosphopeptide comprises an amino acid sequence denoted as Ser-Ser-Ser-Glu-Glu (SEQ ID NO:2), wherein at least one Ser residue, at least two Ser residues or three Ser residues are phosphorylated.

In another embodiment, the phosphopeptide comprises an amino acid sequence denoted as Ser(p)-Ser(p)-Ser(p)-Glu-Glu (SEQ ID NO:3).

In another embodiment, the phosphopeptide comprises an amino acid sequence denoted as RELEELNVPGEIVE S(p)LS(p)S(p)S(p)EESITR (SEQ ID NO:4). In another embodiment, the phosphopeptide comprises an amino acid sequence denoted as QMEAESIS(p)S(p)S(p)EEIVPDSVEQK (SEQ ID NO:5). In another embodiment, the phosphopeptide comprises an amino acid sequence denoted as KNTMEHVS(p)S(p)S(p)EESIISNETYK (SEQ ID NO:6). In another embodiment, the phosphopeptide comprises an amino acid sequence denoted as KVNELSKNIGS(p)ES(p)TEDQ (SEQ ID NO:7). In another embodiment, the phosphopeptide comprises an amino acid sequence denoted as PTLNREQLS(p)TS(p)EENSKKTVD (SEQ ID NO:8). In another embodiment, the phosphopeptide comprises an amino acid sequence denoted as ELEELNVPGEIVES(p)LS(p)S(p)S(p)EESITR (SEQ ID NO:9). In another embodiment, the phosphopeptide comprises an amino acid sequence denoted as RELEELNVPGEIES(p)LS(p)S(p)S(p)EESITR (SEQ ID NO:10). In another embodiment, the phosphopeptide comprises an amino acid sequence denoted as QMEAES(p)IS(p)S(p)S(p)EEIVPNS(p)VEQK (SEQ ID NO:11). In another embodiment, the phosphopeptide comprises an amino acid sequence denoted as KNTMEHVS(p)S(p)S(p)EESIIS(p)QETYK (SEQ ID NO:12). In another embodiment, the phosphopeptide comprises an amino acid sequence denoted as KVNELSKDIGS(p)ES(p)TEDQ (SEQ ID NO:13). In another embodiment, the acid sequence denoted as phosphopeptide comprises an amino ESIIS(p)QETYKQEKNMAINPSKENLCSTFCKEVVRNANEEETSIGS(p)S(p)S(p)EES(p)AEVATEEVKITVDDKHYQKALNEINQFYQKFPGYLQYLYQGPIVLNPWNQVLR NAVPITPTLNREQLS(p)TS(p)EENSKKTVN (SEQ ID NO:14). In another embodiment, the phosphopeptide comprises an amino acid sequence denoted as ELEELNVPGEIES(p)LS(p)S(p)S(p)EESITR (SEQ ID NO:15).

In another embodiment, the phosphopeptide comprises an amino acid sequence denoted as X₁₍ₙ₎-Ser(P)-Ser(P)-Ser(P)-X₂₍ₘ₎-Lys (SEQ ID NO:16), wherein at least one of X₁ and X₂ is independently selected from a positively charged amino acid and wherein each one of n and m is independently selected from 0, 1 and 2.

In some embodiments, the positively charged amino acid is selected from the group consisting of lysine, arginine and histidine. In some embodiments, the positively charged amino acid is lysine. In some other embodiments, the positively charged amino acid is arginine. In some other embodiments, the positively charged amino acid is histidine.

According to some embodiments, the formula of SEQ ID NO:16 further comprises a blocking group (also denoted herein as a protecting group) at the C-terminus. In some embodiments, the carboxyl group at the C terminus of the peptide is protected with a protecting group. The protecting group is selected from, but not limited to an amide (i.e., the hydroxyl group at the C terminus is replaced with a primary amine (NH2), secondary amine, or tertiary amine) or ester (i.e. the hydroxyl group at the C terminus is replaced with an ester). According to some embodiments, the blocking group is selected from the group consisting of amide and ester. According to some embodiments, the blocking group is amide.

In another embodiment, the phosphopeptide comprises an amino acid sequence denoted as Lys-Lys-Ser(P)-Ser(P)-Ser(P) (SEQ ID NO:17). In another embodiment, the phosphopeptide comprises an amino acid sequence denoted as Lys-Lys-Ser(P)-Ser(P)-Ser(P)-Lys (SEQ ID NO:18). In another embodiment, the phosphopeptide comprises an amino acid sequence denoted as Lys-Lys-Ser(P)-Ser(P)-Ser(P)-Lys-Lys (SEQ ID NO:19). In another embodiment, the phosphopeptide comprises an amino acid sequence denoted as Lys-Ser(P)-Ser(P)-Ser(P)-Lys-Lys (SEQ ID NO:20). In another embodiment, the phosphopeptide comprises an amino acid sequence denoted as Lys-Ser(P)-Ser(P)-Ser(P)-Lys (SEQ ID NO:21). In another embodiment, the phosphopeptide comprises an amino acid sequence denoted as Lys-Ser(P)-Ser(P)-Ser(P) (SEQ ID NO:22). In another embodiment, the phosphopeptide comprises an amino acid sequence denoted as Ser(P)-Ser(P)-Ser(P)-Lys-Lys (SEQ ID NO:23). In another embodiment, the phosphopeptide comprises an amino acid sequence denoted as Ser(P)-Ser(P)-Ser(P)-Lys (SEQ ID NO:24). In another embodiment, the phosphopeptide comprises an amino acid sequence denoted as Lys-Lys-Ser(p)-Ser(p)-Ser(p)-NH2 (SEQ ID NO:25). In another embodiment, the phosphopeptide comprises an amino acid sequence denoted as RELEELNVPGEIVES(p)LS(p)S(p)S(p)EESITRINK (SEQ ID NO:26).

The casein-derived peptide according to the invention may comprise “L” as well as “D” form residues. While the amino acid residues of the peptide sequences set forth in SEQ ID NOs: 1-26 are all in the “L” isomeric form, residues in the “D” isomeric form can substitute any L-amino acid residue so long as the resulting peptide analog retains at least part of the biological activity of the corresponding “L” isomer. One reason for designing casein-derived peptides comprising at least one D-amino acid is to increase stability of the peptide to proteolytic degradation.

In one embodiment, the composition is free of antimicrobials and comprises an acceptable carrier. In another embodiment, the composition is further free from hormones, genetic modified organism, or combination thereof. A skilled artisan would understand that a genetically modified organism (GMO) is an animal, plant, or microbe whose DNA has been altered using genetic engineering techniques.

In one embodiment, the milk derived protein is measured by UV at a range from 204 to 220 nm.

### Dosage and Administration

In some embodiments, the methods of the present disclosure comprise administering between 10 ng/ml to 500 mg/ml of the milk derived protein, per administration. In one embodiment, the methods of the present disclosure comprise administering between 1 mg/ml to 500 mg/ml of the milk derived protein, per administration. In another embodiment, the methods of the present disclosure comprise administering between 10 mg/ml to 450 mg/ml of the milk derived protein, per administration. In another embodiment, the methods of the present disclosure comprise administering between 50 mg/ml to 400 mg/ml of the milk derived protein, per administration. In another embodiment, the methods of the present disclosure comprise administering between 50 mg/ml to 70 mg/ml of the milk derived protein, per administration. In another embodiment, the methods of the present disclosure comprise administering between 100 mg/ml to 350 mg/ml of the milk derived protein, per administration. In another embodiment, the methods of the present disclosure comprise administering between 150 mg/ml to 300 mg/ml of the milk derived protein, per administration. In another embodiment, the methods of the present disclosure comprise administering between 200 mg/ml to 250 mg/ml of the milk derived protein, per administration. In another embodiment, the methods of the present disclosure comprise administering between 5 mg/ml to 30 mg/ml of the milk derived protein, per administration.

In one embodiment, the milk derived protein concentration in the composition is between 0.1% to 30%. In another embodiment, the milk derived protein concentration in the composition is 0.1%. In another embodiment, the milk derived protein concentration in the composition is 1%. In another embodiment, the milk derived protein concentration in the composition is 5%. In another embodiment, the milk derived protein concentration in the composition is 10%. In another embodiment, the milk derived protein concentration in the composition is 10%. In another embodiment, the milk derived protein concentration in the composition is 15%. In another embodiment, the milk derived protein concentration in the composition is 20%. In another embodiment, the milk derived protein concentration in the composition is 25%. In another embodiment, the milk derived protein concentration in the composition is 30%.

In one embodiment, the administration comprises oral, intraoral, topical, epicutaneous, transdermal, subcutaneous, intrarectal, intra vaginal, parenteral or any combination thereof. In another embodiment, the administration comprises oral administration. In another embodiment, the administration comprises intraoral administration. In another embodiment, the administration comprises topical administration. In another embodiment, the administration comprises epicutaneous administration. In another embodiment, the administration comprises transdermal administration. In another embodiment, the administration comprises subcutaneous administration. In another embodiment, the administration comprises intrarectal administration. In another embodiment, the administration comprises vaginal administration. In another embodiment, the administration comprises parenteral administration.

In another embodiment, the administration comprises intramammary infusion to a single teat or a plurality of teats. In another embodiment, the administration comprises intramammary infusion to a single teat. In another embodiment, the administration comprises intramammary infusion to a plurality of teats.

In some embodiments, the methods of the present disclosure comprise between one to eight administrations to a single teat. In one embodiment, the methods of the present disclosure comprise one administration. In another embodiment, the methods of the present disclosure comprise two administrations. In another embodiment, the methods of the present disclosure comprise three administrations. In another embodiment, the methods of the present disclosure comprise four administrations. In another embodiment, the methods of the present disclosure comprise five administrations. In another embodiment, the methods of the present disclosure comprise six administrations. In another embodiment, the methods of the present disclosure comprise seven administrations. In another embodiment, the methods of the present disclosure comprise eight administrations.

In some embodiments, the administrations of the present disclosure comprise subsequent administrations. In one embodiment, the administrations are immediately one after the other.

In some embodiments, the administrations of the present disclosure comprise intervals of from about 1 hour to about 72 hours. In one embodiment, the administrations comprise intervals of about 1 hour. In one embodiment, the administrations comprise intervals of about 4 hours. In one embodiment, the administrations comprise intervals of about 5 hours. In one embodiment, the administrations comprise intervals of about 8 hours. In one embodiment, the administrations comprise intervals of about 10 hours. In one embodiment, the administrations comprise intervals of about 12 hours. In one embodiment, the administrations comprise intervals of about 15 hours. In one embodiment, the administrations comprise intervals of about 16 hours. In one embodiment, the administrations comprise intervals of about 20 hours. In one embodiment, the administrations comprise intervals of about 24 hours. In one embodiment, the administrations comprise intervals of about 25 hours. In one embodiment, the administrations comprise intervals of about 28 hours. In one embodiment, the administrations comprise intervals of about 30 hours. In one embodiment, the administrations comprise intervals of about 35 hours. In one embodiment, the administrations comprise intervals of about 36 hours. In one embodiment, the administrations comprise intervals of about 40 hours. In one embodiment, the administrations comprise intervals of about 45 hours. In one embodiment, the administrations comprise intervals of about 50 hours. In one embodiment, the administrations comprise intervals of about 55 hours. In one embodiment, the administrations comprise intervals of about 60 hours. In one embodiment, the administrations comprise intervals of about 65 hours. In one embodiment, the administrations comprise intervals of about 70 hours. In one embodiment, the administrations comprise intervals of about 72 hours.

In one embodiment, the administration comprises intervals of from about 1 hour to about 24 hours.

In one embodiment, the administration is during the lactating period.

In one embodiment, the administration comprises continued administrations during the lactating period or during several lactating periods. In one embodiment the administration comprises continued administrations during the lactating period. In another embodiment the administration comprises continued administrations during several lactating periods. In another embodiment the administration comprises continued administrations during two lactating periods. In another embodiment the administration comprises continued administrations during three lactating periods. In another embodiment the administration comprises continued administrations during four lactating periods. In another embodiment the administration comprises continued administrations during five lactating periods. In another embodiment the administration comprises continued administrations during six lactating periods. In another embodiment the administration comprises continued administrations during seven lactating periods. In another embodiment the administration comprises continued administrations during eight lactating periods. In another embodiment the administration comprises continued administrations during nine lactating periods. In another embodiment the administration comprises continued administrations during ten lactating periods.

In one embodiment, the administration comprises continued administrations during the dry-off period or during several dry-off periods. In one embodiment the administration comprises continued administrations during the dry-off period. In another embodiment the administration comprises continued administrations during several dry-off periods. In another embodiment the administration comprises continued administrations during two dry-off periods. In another embodiment the administration comprises continued administrations during three dry-off periods. In another embodiment the administration comprises continued administrations during four dry-off periods. In another embodiment the administration comprises continued administrations during five dry-off periods. In another embodiment the administration comprises continued administrations during six dry-off periods. In another embodiment the administration comprises continued administrations during seven dry-off periods. In another embodiment the administration comprises continued administrations during eight dry-off periods. In another embodiment the administration comprises continued administrations during nine dry-off periods. In another embodiment the administration comprises continued administrations during ten dry-off periods.

### EXAMPLES

### Example 1—Casein Hydrolysate by Intramammary Administration as a Dry Cow Therapy

The objective of this randomized, blinded, controlled study was to assess the association of intramammary administration of bovine casein hydrolysate (bCNH) with milk production in subsequent lactation and protein, butterfat, and lactose yields, also in subsequent lactation. Milk production was measured by milk yield, by energy corrected milk (ECM), combining protein, butterfat, and lactose yields. During the subsequent lactation. local tolerability of the infusion and systemic conditions were assessed as well.

Total 306 enrolled Israel Holstein Friesians dairy cows were included in the analysis. Eligibility criteria included good general condition, no clinical mastitis or any other intramammary infections. Fourteen (14) cows were excluded from the total eligible and enrolled dairy cows in the study due to management events (5), abortion (6), and mammary conditions (3). Enrolment of cows was according to routine farm entry to the pre-dry period.

There were two bCNH experimental groups and one positive control group. The experimental groups included as follows: (i) 109 cows treated with a single syringe (20 mL of 1200 mg bCNH), (bCNH-11) (ii) 96 cows treated consecutively with two syringes of 20 mL each (40 m mL), (bCNH-22). The control group, which consisted of 101 cases, was treated with conventional dry cow therapy (DCT) Nefpenzal® DC.

### Analysis of Energy Corrected Milk (ECM)

Energy-corrected milk (ECM) determines the amount of energy in the milk based on the milk composition, specifically fat and protein. Calculating ECM is used to measure the feed efficiency (FE) of a cow's ability to convert dry matter feed into milk yield (kg) by dividing the ECM by the amount of dry matter intake. Feed efficiency is used for genetic evaluation, and the effect of Temperature-Humidity Index.

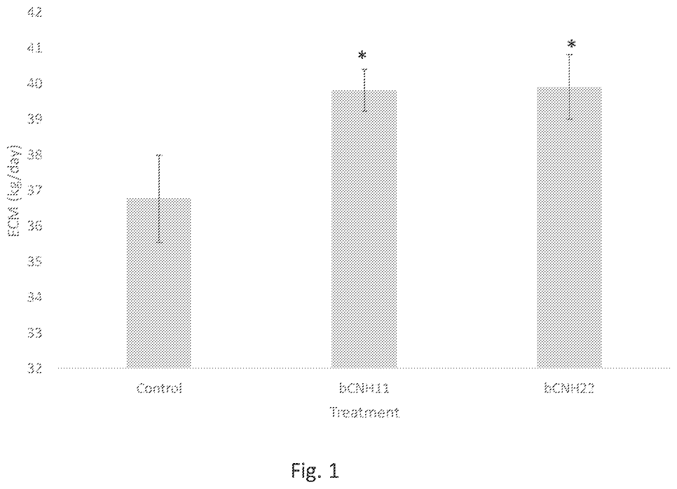

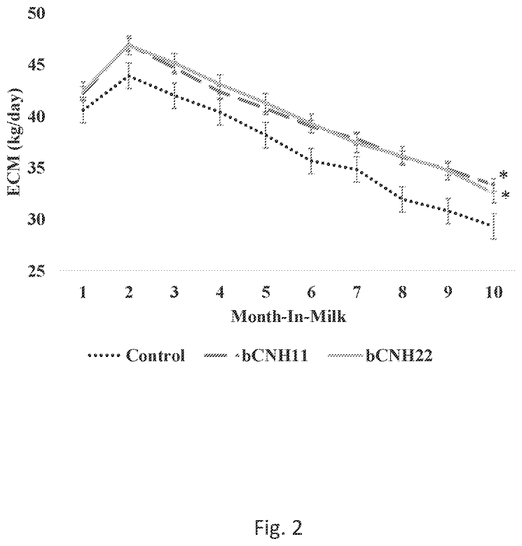

The results of the ECM production (kg) by month in milk along 305-days of subsequent lactation are presented in FIG. 1 and FIG. 2. The analysis of difference between each of bCNH treatment groups and control revealed that bCNH11 and bCNH22 were significantly different from control (P<0.05)). The ECM yield of bCNH11 was higher by 3.07 kg per day than the control over the 305-days of lactation, and that of bCNH22 was higher by 2.96 kg than in the control group. For the whole period of 305-days of lactation, the differences between control and these bCNH groups were 965 kg for bCNH11 and 902 kg for bCNH22 per cow. The average ECM yield of bCNH treatments was higher than that of control along all timepoints of 305-days of lactation.

### Analysis of Butterfat Yield

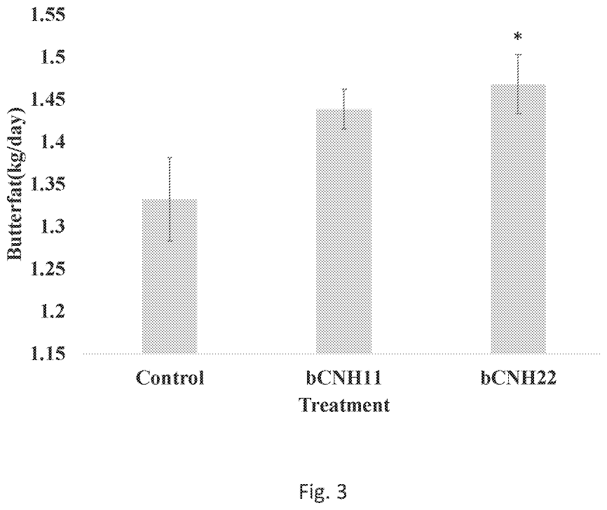

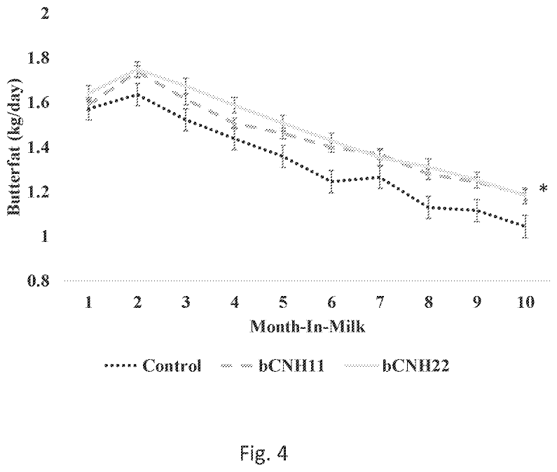

The results of the linear mixed model for butterfat yield (kg) by month along 305-days of subsequent lactation are presented in FIG. 3 and FIG. 4.

### Analysis of Protein Yield

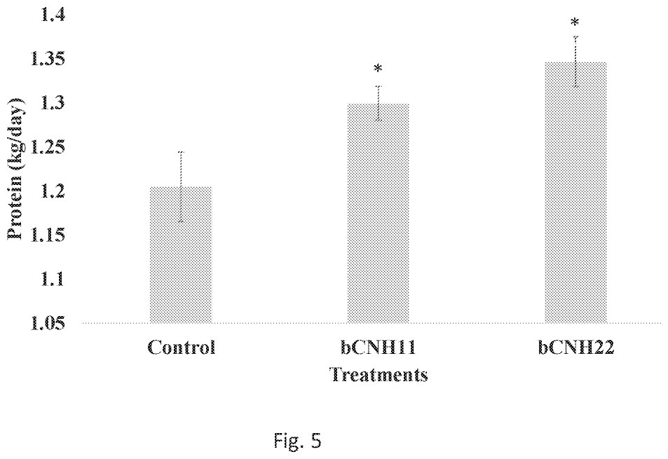

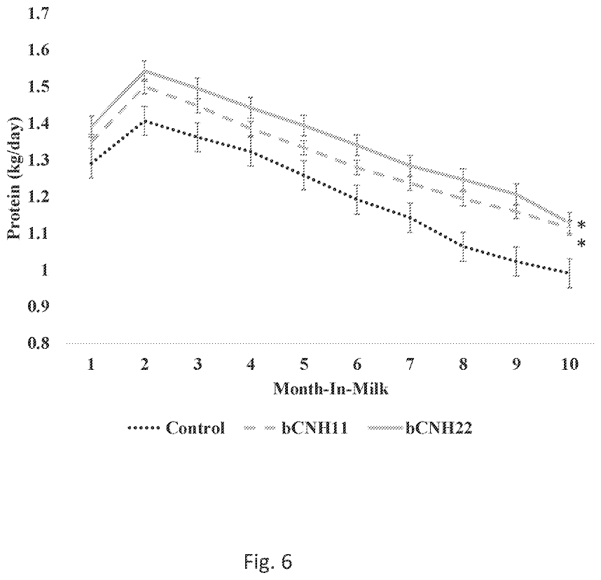

The results of the linear mixed model for protein yield (kg) by month along 305-days of subsequent lactation are presented in FIG. 5 and FIG. 6.

### Analysis of Lactose Yield

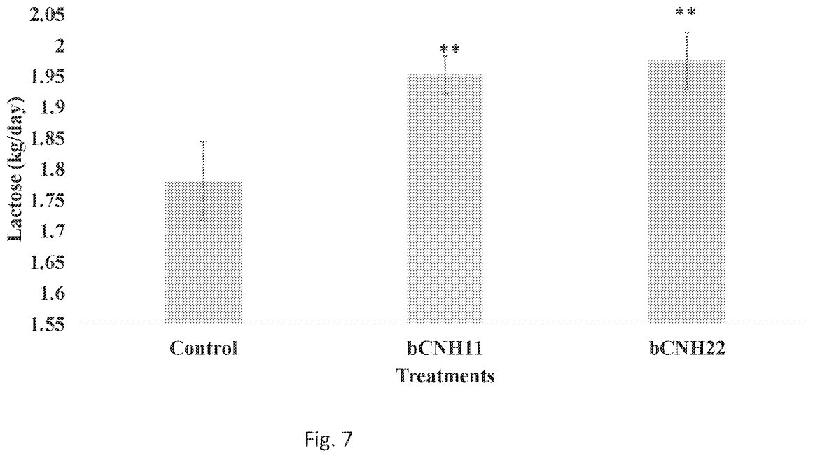

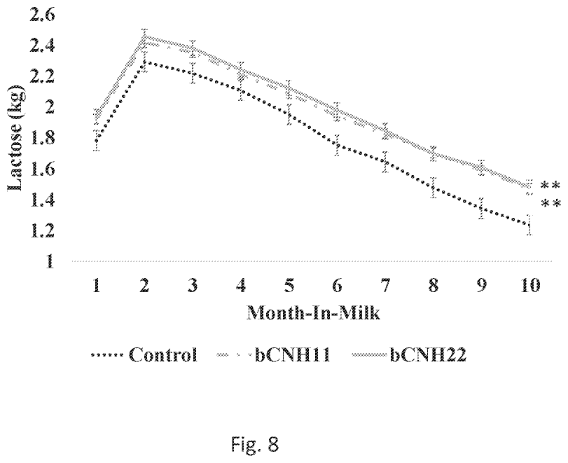

The results of the lactose yield by month along 305-days of subsequent lactation are presented in FIG. 7 and FIG. 8.

The above results of Energy Corrected Milk and milk biological components (mainly protein, butterfat and lactose) enhancement during lactation after drying cattle with bCNH are innovative and surprising findings. Treating with bCNH did not have any negative effect on milk yield compared with antibiotics. Furthermore, the significance of higher butterfat and protein yields, expressed later by higher ECM, strengthens the idea that bCNH is very effective as dry-off treatment.

### Example 2—Field Study to Evaluate the Effect of Bovine Casein Hydrolysate on Milk Quality, During the Lactation Period

The currently field study aim was to evaluate the clinical benefit of milk yield and milk properties of bCNH intramammary infusion to quarters during the lactation period and after reinitiation of milking (following a period of five days of non-milking following administration of bCNH).

Twenty-three pregnant (23) Israeli Holstein-Friesian dairy cows of any parity were enrolled after clinical examination to confirm subclinical mastitis, as determined by elevated SCC and after two consecutive bacteriology tests positive to NAS during the screening period (Study Days −7 and −6). On treatment day (Day 0), after clinical examination and following morning milking, eligible cows (infected quarters, 1 or 2 per cow) were randomized to either receive bCNH or be untreated (negative control). After bCNH treatment, the treated quarters had a non-milking period of 6 (±1) days, followed by reinitiation of milking. Other quarters, either control (untreated) or not included in the study (not infected), continued to be milked per normal milking management process. Milk samples were collected from the infected udder quarters on the treatment day (D 0) before treatment, on the reinitiation day (D 6±1), twelve hours after, subsequent morning milking-day, and 14- and 21-days post-treatment, for bacteriology, somatic cell count (SCC), lactose, sodium:potassium ratio, and conductivity of milk. Following treatment, all cows were managed per usual management operation. All cows were monitored for clinical signs of mastitis or other diseases up to study day 21. Daily milk yield data was collected from the NOA system (the Israeli Dairy Herd Management Program developed by the Israeli Dairy Board) until day 35 post treatment. Additionally, routine monthly data on milk yield and milk components was collected for the period of up to three months before the study entry (treatment) and up to three months afterwards.

Each cow in the study received treatment administered to the infected udder quarter(s) only, i.e., the infected quarter in each cow received either 1200 mg bCNH (T2), or 2400 mg bCNH (T3), or 4800 mg bCNH (T4), or did not receive any treatment (T1).

Treated udder quarter(s) from each participant cow were not milked during 6 (+1) consecutive days and initiated re-milking afterwards. From the treated cow, untreated quarters were normally milked, and their milk was not discarded.

The effect of treatment on milk yield, Energy Corrected Milk (ECM), butterfat (kg), protein (kg), lactose (kg), and composite SCC was tested.

Monthly milk yield, ECM, butterfat, protein, lactose, and composite SCC data were collected from 3 months before treating the cows, and 3 months after reinitiating of milking. Linear mixed model, using JMP 16.2.0, LSmeans differences Student's t, was used to analyze the difference between the control and active treatment group. The model includes parity (1, 2, 3+), month-in-milk after reinitiating milking (MIM), treatment (control, T2), interactions of treatment by MIM and by parity. The average of each independent variable of the period before entry to the trial was calculated and was used as a covariate.

Energy Corrected Milk (ECM) Values (Kg) for Three Months Pre- and Post Re-Milking periods

Energy corrected milk (kg) was calculated according to the following formula:

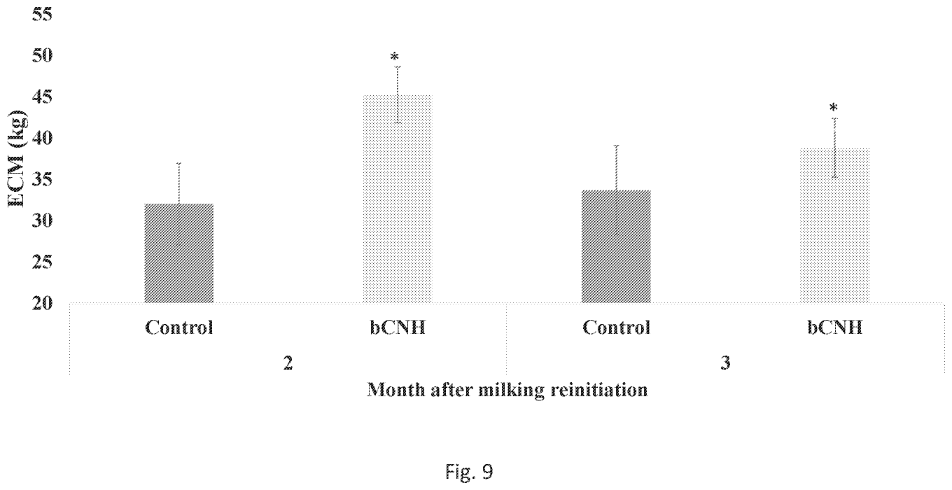

ECM=0.1*milk+milk*butterfat %/100*10*+milk*protein %/100*15.8. Average of monthly ECM before entry to the trial was used as a covariate. Energy corrected milk yield of control group for the whole period of post-milking reinitiation was 35.88 (SE=4.06) and of bCNH group was 39.86 (SE=2.76), P=0.43. As can be seen in FIG. 9 a significant difference was observed in the second month.

### Composite Protein Values (Kg) in Milk for 35-Days Pre- and Post Re-Milking Periods

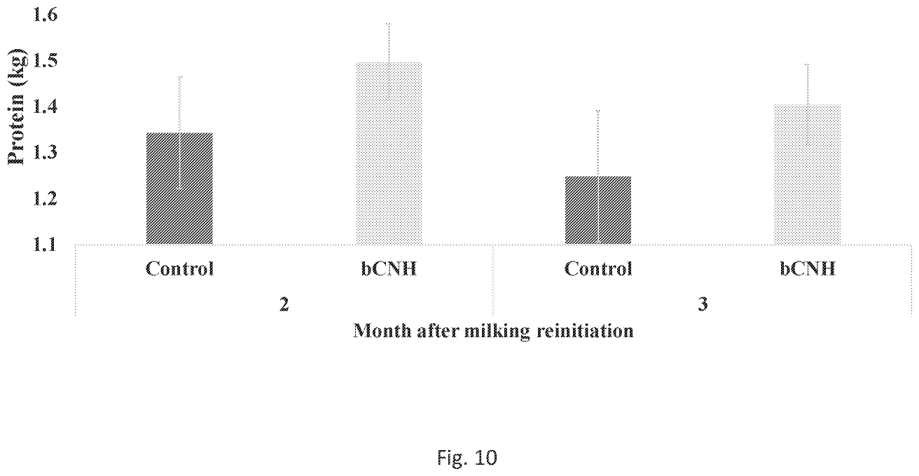

Average of monthly protein yield before entry to the trial was used as a covariate. The protein yield of control group for the whole 3 months period after initiation of milking was 1.32 kg (SE=0.098) and of bCNH group-1.38 kg (SE=0.065), P=0.08. As can be seen in FIG. 10, the protein yield became higher in 2_{nd} and 3_{rd} month.

### Composite Butterfat Values (Kg) in Milk for 35-Days Pre- and Post Re-Milking Periods

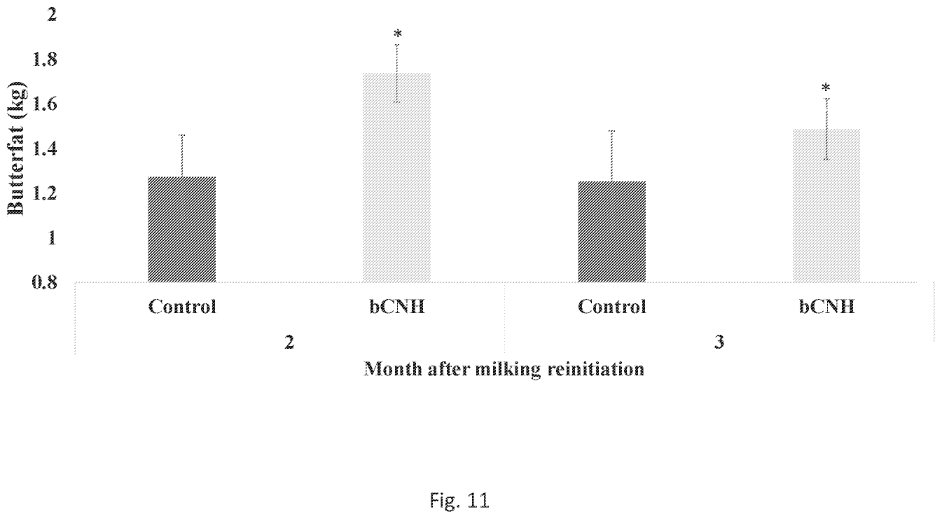

Average of monthly butterfat yield before entry to the trial was used as a covariate. Butterfat yield of control group was 1.36 kg (SE-0.14) and of bCNH group-1.49 (SE=0.09), (P=0.46). As can be seen in FIG. 11, butterfat yield became higher during 2nd and 3rd months measurements.

### Composite Lactose Values (Kg) in Milk for 35 Days Pre- and Post Re-Milking Periods

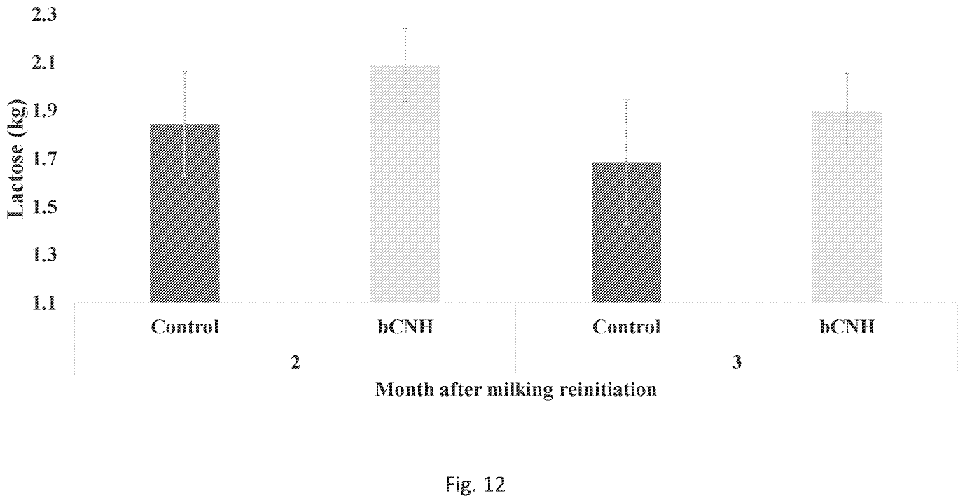

Average monthly lactose yield before entry to the trial was used as a covariate. Least Square mean of lactose yield of control group was 1.82 (SE=0.18) and of bCNH group-1.90 (SE=0.12). As can be seen in FIG. 12, lactose yield became higher in months 2 and 3 post treatment.

Composite—Bulk Milk Somatic Cell Count (BMSCC) During the Pre- and Post-Re-Milking periods

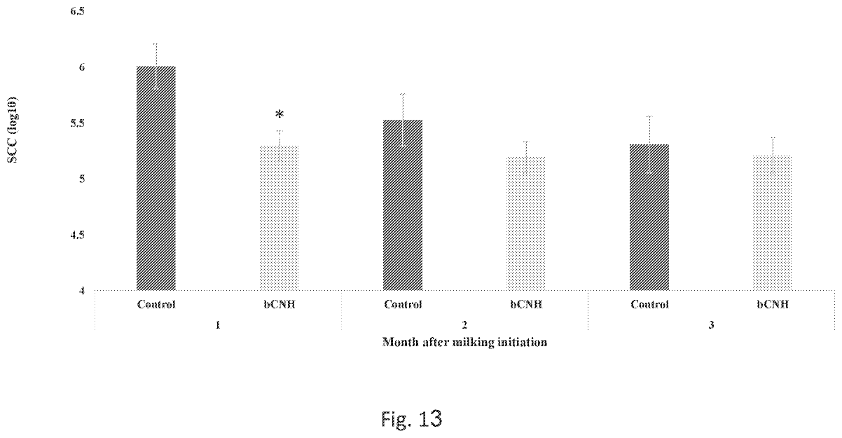

Bulk milk somatic cell count values are not normally distributed (Prob>chiSq=0.026). Therefore, BMSCC values were transformed to log 10. In order to understand the effect of treatment on log₁₀BMSCC after milking reinitiation independently of the pre-treatment levels, the log₁₀BMSCC before treatment was used as a covariate. Log₁₀BMSCC of control group for whole post-remilking period was 5.58 (SE-0.18) and of bCNH group-5.14 (SE=0.13), P=0.051. The differences in each Month-In-Milk (MIM) revealed that log 10BMSCC of control cows was significantly higher than that of experimental group (P=0.005) in the first month post milking initiation (FIG. 13).

### Monthly Milk Yield Values (kg) for 35-Days Pre- and Post Re-Milking Periods

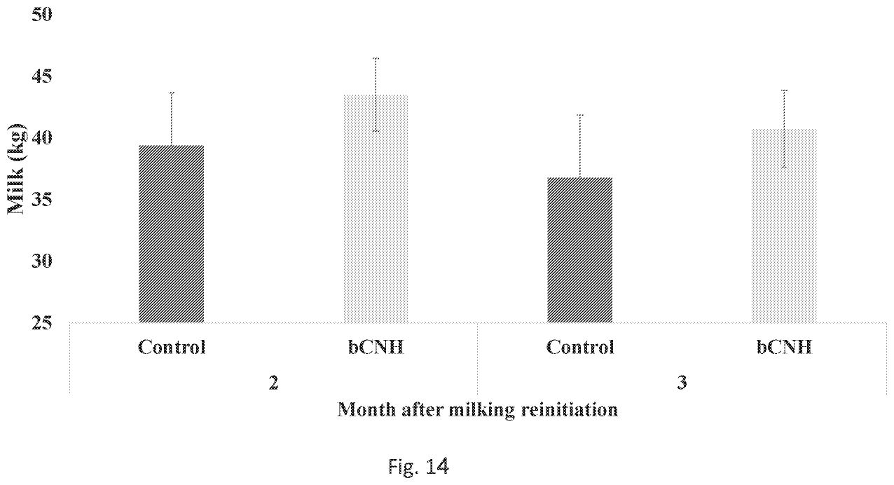

Monthly milk yield of control group for the whole period of post-milking reinitiation was 39.27 kg (SE=3.56) per day on average, and of bCNH group-39.92 kg (SE=2.37, P=0.88) per day on average. As can be seen in FIG. 14, higher average milk yield was observed for the bCNH treatment group. It therefore seems that there is a tendency for higher milk yield over time in the experimental (bCNH) group.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method of increasing milk quality in lactating mammals comprising administering a composition comprising at least one casein derived peptide.

2. The method according to claim 1, wherein said increased milk quality comprises increased milk protein, increased milk butterfat, increased milk lactose, increased energy corrected milk (ECM) or any combination thereof.

3. (canceled)

4. (canceled)

5. (canceled)

6. (canceled)

7. The method according to claim 2, wherein the milk protein percentage in the milk is in the range of 2.5%-6.5%.

8. The method according to claim 2, wherein the milk protein percentage in the milk is increased by 0.5%-30% in comparison to milk protein in control lactating mammals.

9. (canceled)

10. (canceled)

11. (canceled)

12. The method according to claim 2, wherein said milk butterfat percentage in the milk is in the range of 2.5%-6.5%.

13. The method according to claim 2, wherein said milk butterfat percentage in the milk is increased by 0.5%-30% in comparison to milk butterfat in control lactating mammals.

14. (canceled)

15. (canceled)

16. (canceled)

17. The method according to claim 2, wherein said milk lactose percentage in the milk is in the range of 3.5-7.8%.

18. The method according to claim 2, wherein said milk lactose percentage in the milk is increased by 0.5%-30% in comparison to milk lactose in control lactating mammals.

19. (canceled)

20. (canceled)

21. (canceled)

22. The method according to claim 2, wherein said ECM is increased by 0.5%-20% in comparison to ECM in control lactating mammals.

23. (canceled)

24. (canceled)

25. The method according to claim 1, wherein said milk quality further comprises decrease in somatic cell count (SCC).

26. The method according to claim 1, wherein said milk quality is increased at any stage of the lactating cycle.

27. (canceled)

28. The method according to claim 1, wherein said milk quality is increased during the same lactation of administration of said composition comprising at least one casein derived peptide or during the following lactation of administration of said composition comprising at least one casein derived peptide.

29. (canceled)

30. (canceled)

31. The method according to claim 1, wherein said milk quality is increased, independent of the dry period length.

32. (canceled)

33. (canceled)

34. (canceled)

35. (canceled)

36. The method according to claim 1, wherein said casein derived peptides comprises natural peptide, synthetic peptide, semi-synthetic peptide, or any combination thereof.

37. (canceled)

38. (canceled)

39. (canceled)

40. The method according to claim 1, wherein said casein derived peptide comprises one or more fragments of β-casein, αS1-casein, αS2-casein, κ-casein, optimally further comprises amino acids with different lengths or any combination thereof.

41. The method according to claim 1, wherein said casein derived peptide comprises a casein hydrolysate.

42. The method according to claim 1, wherein said casein derived peptide comprises a phosphopeptide.

43. The method according to claim 42, wherein said phosphopeptide comprises an amino acid sequence selected from the group consisting of SEQ ID NO. 1-SEQ ID NO 26.

44. (canceled)

45. (canceled)

46. (canceled)

47. (canceled)

48. (canceled)

49. The method according to claim 1, wherein said administration comprises continued administrations during the lactating period or during several lactating periods.

50. The method according to claim 1, wherein said milk is essentially free of residues and wherein said milk can be used as a raw milk, for dairy production, for breastfeeding, bakery, confectionary, feeding or any combination thereof.

51. (canceled)

52. (canceled)
